(19) United States
(12) Patent Application Publication
(10) Pub. No.: US 2008/0020019 A1
(43) Pub. Date: Jan. 24, 2008

(54) METHOD OF DECREASING FAT DEPOSITS AND BODY WEIGHT IN MAMMALS AND BIRDS

(76) Inventor: A.K. Gunnar Aberg, Sarasota, FL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/834,580

(22) Filed: Aug. 6, 2007

Related U.S. Application Data
(63) Continuation of application No. 10/987,665, filed on Nov. 12, 2004, now abandoned.
(60) Provisional application No. 60/524,376, filed on Nov. 20, 2003.

(51) Int. Cl. A61K 31/133; A61K 9/00; A61P 3/04

(52) U.S. Cl. 424/439; 514/653

(57) ABSTRACT

A method is disclosed utilizing an optically pure eutomer of salbutamol for reducing body fat and/or body weight in mammals and birds. A food composition including the eutomer is also disclosed.

This application claims priority of provisional patent application Ser. No. 60/524,376 filed Nov. 20, 2003, the disclosure of which is hereby incorporated by reference.

### BACKGROUND OF THE INVENTION

Many biologically active molecules exist as enantiomers. Although structurally identical, enantiomers can have different effects in biological systems: one isomer may have specific therapeutic activity while the other isomer may have no therapeutic activity or may have entirely different forms of biological activity.

Salbutamol is called albuterol in the United States of America.

Adrenergic beta-2 receptor agonist drugs (also called beta-2 agonists) are presently used as racemic mixtures of isomers. As an example, racemic salbutamol is a mixture containing 50 percent R-salbutamol and 50 percent S-salbutamol. An R-isomer is structurally identical to the corresponding S-isomer and the isomers differ only in that one isomer is a mirror image of the other. Molecules with two chiral centers have four isomers, ex. RR-formoterol, SS-formoterol, RS-formoterol and SR-formoterol. Racemic formoterol contains all four isomers. The therapeutically active isomers (the eutomers) of beta-2 agonists have affinity for the beta-2 receptors in the body and are usually the R- or RR-isomers, while the S- or SS-isomers usually do not carry adrenergic activity (distomers). Exceptions from this general rule exist and an example is salmeterol, where both isomers have affinity for adrenergic beta-receptors.

Most adrenergic beta-receptor agonists have affinity for various types of beta receptors. Thus, as shown below, salbutamol has affinity for beta-1 and beta-2 receptors, but not for beta-3 receptors.

| | Affinity for receptor (Ki)*) | | |
|---|---|---|---|
| Compound | Beta-1 (nM) | Beta-2 (nM) | Beta-3 (nM) |
| RS-salbutamol | 2,980 | 668 | >>10,000 |
| R-salbutamol | 1,540 | 236 | >>10,000 |
| S-salbutamol | >>10,000 | >>10,000 | >>10,000 |

*)Results regarding Beta-1 and Beta-2 were obtained from Penn et al. 1996. Results regarding Beta-3 receptor affinity were obtained from BRIDGE PHARMA, Inc., Sarasota, Florida, USA.

Contrary to the beta-receptor agonist salbutamol, the beta-receptor agonist clenbuterol has affinity for all three adrenergic beta-receptors: beta-1, beta-2 and beta-3.

The therapeutic activity of a beta-2 agonist like salbutamol is to activate adrenergic beta-2 receptors and thereby initiate cellular responses, the most well-known of which is the relaxation of bronchial smooth muscles. Adrenergic beta-2 agonists also have metabolic effects, such as for example lipolytic effects, but the lipolytic effects of beta-2 receptor agonists have been described as weak or even minimal. The lipolytic effect of beta-receptor activation is believed to be caused mainly by activation of adrenergic beta-3 receptors.

It has now been found that oral administration of low doses of R-salbutamol cause a significant loss of body fat, which is surprising and difficult to explain since R-salbutamol does not have affinity for adrenergic beta-3 receptors. It can be speculated that R-salbutamol may have central effects within the CNS, possibly through affinity for melanocortin-4 receptors. However, although the reduction of body fat by R-salbutamol may not exclusively be an effect on adrenergic beta-receptors, this activity is stereoselectively residing in the R-isomer of salbutamol.

The pharmacological effects, the metabolism and the toxicity of adrenergic beta-agonists vary, depending on the animal species and which drug is studied.

It has now surprisingly been found that the R-isomer of salbutamol is potently and stereoselectively reducing the fat content in the bodies of mammals and birds.

The S-isomer of salbutamol is inactive in this respect and did not reduce fat. However, we have shown that R- and S-salbutamol have the same acute toxicity when tested in mammals. The S-isomer of salbutamol also causes smooth muscle hyperreactivity, which is a serious and stereoselective side effect of S-salbutamol (Yamaguchi et al. 1996)

The most commonly used therapeutic indication for beta-agonists in man is to treat bronchial spasms in asthmatic individuals or in individuals suffering from bronchitis. R-salbutamol has also been shown to induce bronchial relaxation and to inhibit premature contractions of the pregnant uterus in humans and to act as a growth promoter in livestock.

### SUMMARY OF THE INVENTION

The present invention relates to a method of decreasing body fat and/or body weight of mammals, such as for example cattle, swine, horses, sheep, deer, dogs, cats and humans and of birds, such as for example turkeys, chicken, ducks and geese, by administering the pure or substantially pure R-isomer of the adrenergic beta-2 receptor agonist salbutamol, while avoiding the toxicity and the side effects residing in the S-isomer of the drug. The method is particularly useful in overweight or obese mammals, including humans and livestock species with high body content of fat, in which the amount of body fat is significantly reduced by the drug. The invention also relates to a food composition including an admixture of protein-containing food materials with the optically pure R-isomer of the adrenergic beta-2 receptor agonist salbutamol or a pharmaceutically acceptable salt thereof, the R-isomer of salbutamol being substantially free of the corresponding distomeric S-isomer.

The invention is based on the surprising observation that administration of R-salbutamol causes a very significant loss of body fat and body weight, which is surprising and difficult to explain since this molecule has minimal or no affinity for adrenergic beta-3 receptors. Thus, the lipolytic activity of R-salbutamol at least equals the lipolytic activity of combined beta-2 and beta-3 agonists, such as for example clenbuterol. It can be speculated (although the present invention is not to be limited thereby) that R-salbutamol causes lipolytic activity through central effects within the CNS, possibly through affinity for melanocortin-4 receptors, although such effects have not been described in the prior art. Furthermore, it has now surprisingly been found that weight loss is achieved even when the drug is administered only once or twice daily. There is no correlation between the adrenergic beta-2 effects of R-salbutamol on heart rate, which is of short duration and the effects of the compound on body weight, which is developing over time. Regardless of the mode(s) of activity of R-salbutamol, the lipolytic activity of this compound is stereoselectively residing in the R-isomer of the compound.

In cases of chicken, turkeys, cows, pigs, sheep, farmed deer and farmed fish and of other livestock animals that enter the food chain, there is a risk that drugs consumed by these animals can induce side effects in people eating the meat after those animals have been slaughtered. However, after oral administration the plasma half-life of R-salbutamol is significantly shorter than the composite plasma half-life of R-salbutamol. (Boulton et al. 1996; Fawcett et al. 1997). Moreover, the low therapeutic doses of the drug, though effective for weight loss, lowers the risk of side effects in humans, consuming the meat of livestock animals that have been dosed with R-salbutamol.

Since the R-isomer of salbutamol does not carry such side effects as bronchial hyperreactivity, increased intraocular pressure, increased uterine contractility or teratogenic activity, the R-isomer is preferred rather than the racemic mixtures of the compound. It is also preferred for toxicological reasons. Thus, the present invention provides a safe, effective method for treating mammals, such as livestock animals, companion animals and humans, with the therapeutically active isomer of salbutamol, the purpose of such treatment being the reduction of the fat content and/or body weight of said mammal.

It has also surprisingly been found that by administration of the pure R-isomer of salbutamol to livestock animals, much or all of the mental agitation seen in the animals after administration of racemic adrenergic beta-agonists, such as for example ractopamine, is avoided.

### DETAILED DESCRIPTION OF THE INVENTION

The R-isomer of salbutamol has now been found to very significantly reduce body fat content and body weight in birds and mammals, particularly in overweight or obese mammals. The corresponding S-isomer has no such activity. The acute toxicity of the therapeutically inactive S-isomer of salbutamol (LD50; iv mice: <60 mg/kg) was found to be similar to the acute toxicity of the therapeutically active R-isomer (LD50; iv mice: <60 mg/kg). Thus, the S-isomer of salbutamol has toxicological activity, but not therapeutic activity. S-salbutamol has also been found to cause serious pharmacological side effects, such as for example hyperreactivity of bronchial and uterine smooth muscle.

The present invention relies on the activity of the beta-2 receptor agonist R-salbutamol to provide decreased body content of fat, while simultaneously avoiding the side effects that are caused by the Sisomer of said adrenergic beta-receptor agonist. Thus, in the present method, a pure or substantially pure eutomer of salbutamol, substantially free of its corresponding S-isomers, can be administered alone, or in combination with at least one other drug in adjunctive treatment, to birds such as turkeys, chicken, ducks and geese, and mammals, such as for example cattle, swine, horses, sheep, deer, dogs, cats and humans, in whom a decrease in body fat and/or body weight is desired. The R-isomer of salbutamol as used herein refers to the optically pure R(+)-isomer of α′[(tert-butylamino) methyl]-4-hydroxy-m-xylene-α,α′-diol, and to any biologically acceptable salt or ester thereof. Clenbuterol refers to 4-amino-α-[(tert-butylamino)methyl]-3,5-dichlorobenzyl alcohol, and to any biologically acceptable salt or ester thereof; salmeterol refers 4-hydroxy-α′-[[[6-(4-phenylbutoxy)-hexyl]amino]methyl]-m-xylene-α,α′-diol and to any biologically acceptable salt or ester thereof, and terbutaline refers to 1-(3,5-dihydroxy-phenyl)-2-(tert-butylamino)ethanol and to any biologically acceptable salt or ester thereof.

Optically pure and substantially pure adrenergic beta-agonists are readily obtainable by methods known to those skilled in the art, e.g., by resolution of a synthetic intermediate or resolution of racemic salbutamol (Hamied et al. WO 02/48090; Gao et al. U.S. Pat. No. 5,399,765; Gao et al. U.S. Pat. No. 5,545,745; Ferrayoli et al., 2000) into its isomers.

The term “substantially pure” as used herein means that at least about 85% of the eutomer and 15% or less of the distomer is present, preferably at least about 95% of the eutomer is present, most preferably at least about 98% of the eutomer is present.

In the present method, the active single isomer or isomers of an adrenergic beta-receptor agonist, such as, but not limited to salbutamol, clenbuterol, formoterol, ractopamine, salmeterol or terbutaline is administered to a bird or mammal, in which decreased body content of fat and/or the reduction in body weight is desired. For example, R-salbutamol is administered to an over-weight or obese human in order to decrease the content of body fat and/or reduce body weight without causing increased risk of bronchial side effects or CNS side effects caused by the corresponding S-isomer.

In the present method, the eutomeric form of salbutamol can be administered by any suitable means, including parenterally, transdermally, subcutaneously, intravenously, intramuscularly or orally, topically, rectally, by inhalation or via implanted reservoirs containing the drug. The form in which the drug will be administered (e.g. inhalant, powder, granulate, tablet, capsule, solution, emulsion, transdermal patch, etc.) will depend on the route by which it is administered. The preferred route of administration is the oral route, with the eutomer of salbutamol mixed into the feed of birds or animals or administered in tablet or a similar form to humans. Tablets for oral administration can be one of the many available controlled-release type tablets or capsules. Additionally, other preferred forms of administration are by inhalation or by transdermal patch, which will reduce or avoid gastrointestinal metabolism and hepatic first-pass metabolism by sulfotransferases and other metabolizing enzymes; such delivery systems may be designed to delay the absorption rate and therefore decrease the maximal plasma drug concentration (Cₘₐₓ).

The quantity of the drug to be administered to individual human patients or to a specific animal will be determined on an individual basis, and will be based on the pharmacological potency of the drug in the selected species, the route of administration, the size of the mammal or bird and the results sought. In general, quantities of the eutomeric drug sufficient to decrease body fat and/or reduce body weight will be administered. The actual dosage (quantity administered at a time) and the number of administrations per day will depend on the pharmacokinetic property of the drug and the metabolism of the drug in the body of the specific mammal species. For example about 10 to 3000 micrograms of the optically pure R-isomer of salbutamol may be given by various forms of inhalation devices, such as metered dose inhalers and nebulizers, 0.01 to 200 milligrams may be given by the oral route (for example as powders, granulates, tablets or liquids) one to four times per day (or ad lib to animals) and may be an adequate dose in most mammals to produce the desired effect. Suitable oral doses in humans include doses in the range of 0.5 mg to 5 mg from once daily to up to four times daily. Suitable doses to animals can be administered in the food material, wherein the concentration of the R-isomer of salbutamol in said food materials may range from approximately 1 ppm to approximately 20 ppm by weight.

The doses of R-salmeterol, R-clenbuterol, RR-formoterol or RR-ractopamine may be lower and the dosing can also be less frequent than is the case with R-salbutamol. Drug doses may be higher or lower and administration may take place more or less frequently than indicated above, as determined by the caring physician or veterinarian.

In the method of the present invention, the eutomer of the beta-2 receptor agonist salbutamol can be prepared as a tablet or a similar formulation to be taken orally by humans in need thereof. The drug can also be administered in a transdermal patch formulation.

In the method of the present invention, the eutomer of the beta-2 receptor agonist salbutamol can be prepared as a dry powder or granulate and mixed into the feed of mammals. The drug can be pre-mixed into a concentrate according to any of the methods known to those skilled in the art or may be mixed into the ultimate animal feed in connection with the actual feeding. The drug may also be administered to animal in the drinking water or by use of an implanted or partially implanted device that delivers the drug parenterally to the animal. When administered to humans, the drug of the present invention can be administered in a capsule, tablet, granulate, powder, or liquid, mist, aerosol, injection, etc. The components included in a particular formulation—in addition to the drug of the present invention—are determined primarily by the manner in which the composition is to be administered. For example, a composition to be administered in liquid form can include, in addition to the drug(s), a liquid carrier, an emulsifying agent, a flavoring agent, an antibacterial or a bacteriostatic agent and/or a coloring agent. A formulation to be administered in powder form or as a granulate can include a filler (e.g., lactose), a binder (e.g., carboxymethyl cellulose, gum arabic, gelatin), an adjuvant, a flavoring agent, and/or a coloring agent. The method used to prepare tablets, capsules, syrups, inhalation solutions, transdermal delivery systems (patches) are all well known to those skilled in the art of preparing pharmaceutical formulations.

The selected compound of the present invention (R-salbutamol) has low toxicity and few side effects and may be administered chronically for a prolonged period of time, said period being for life, for multiple years, or for as long period as the therapeutic activity is deemed necessary.

Known side effects of R-salbutamol include tachycardia and tremor. These and other side effects are of short duration and are associated with high plasma concentrations of the drug. These side effects can be reduced or completely avoided by using drug delivery systems that slowly release the drug of the present invention into the systemic circulation. Such slow-release delivery systems include tablets or capsules that are designed to slowly release the active ingredient.

In the method of the present invention, the R-isomer of the selected drug can be administered together with one or more other active compound(s). Compounds that improve or prolong the therapeutic effect of beta-2 agonists, e.g. compounds that delay or inhibit the absorption or the metabolic degradation of the compound (for example acetaminophen or phosphodiesterase inhibitors), may also be co-administered with the eutomeric beta agonist to further improve the therapeutic activity. Other drugs than R-salbutamol that cause fat loss or weight loss may be combined with the selected drug of the present invention to obtain improved therapeutic activity and further reduce body fat or body weight.

The eutomeric receptor agonists of the present invention can be administered together with an adrenergic beta-receptor blocker, said beta-receptor blocker administered in a dose, way or form that inhibits or reduces beta-receptor mediated side effects, but not the therapeutic activities of fat loss or weight loss.

Fat loss therapy or weight loss therapy using an adrenergic beta-agonist can and should be combined with appropriate life-style modifications, such as for example modified eating habits and increased exercise.

The two (or more) drugs (the optically pure active drug of the present invention, together with the other drug(s)) can be administered in one composition or as separate entities. For example, they can be administered in a single capsule, tablet, granulate, powder, or liquid, mist, aerosol, injection, etc. or as individual drug formulations. The components included in a particular formulation—in addition to the drug of the present invention and another drug or drugs—are determined primarily by the manner in which the composition is to be administered. For example, a composition to be administered in liquid form can include, in addition to the drug(s), a liquid carrier, an emulsifying agent, a flavoring agent, an antibacterial or a bacteriostatic agent and/or a coloring agent. A formulation to be administered in powder form or as a granulate can include a filler (e.g., lactose), a binder (e.g., carboxymethyl cellulose, gum arabic, gelatin), an adjuvant, a flavoring agent, and/or a coloring agent.

In general, according to the method of the present invention, the optically active eutomer of salbutamol, alone or in combination with another drug(s), is administered to animals, birds or humans, periodically or continuously as necessary to reduce body fat and/or reduce body weight.

The present composition and method provide effective treatment while eliminating the undesired side effects induced by the administration of the distomer in racemic salbutamol, in humans given the drug and in humans eating meat from drug-treated livestock animals. These side effect include but are not limited to bronchial hyperreactivity, increased uterine contractility, increased intraocular pressure, central nervous system effects such as aggression, tremor, shakiness, and dizziness, and cardiovascular side effects, induced by the S-isomer in racemic salbutamol and the racemic mixtures of other beta-2 agonists. Thus, by the administration of the pure R-isomer of salbutamol, the side effects of the corresponding distomer, which can be of prolonged duration, will be reduced or avoided.

Biological Effects

Background

It is known from the prior art that various racemic beta-2 agonists can be used to improve performance of livestock animals. The improved performance includes increased efficiency (gram feed eaten per gram gain of body weight) and increased muscle weight. However, the reported decrease of fat by beta-agonists in livestock animals, including poultry, has been described as modest or non-existing. Thus, in experiments on chicken, Dalrymple et al (Poultry Science, 1984, 63: 2376-2383) reported that the beta-2 agonist clenbuterol did not reduce the abdominal fat pad in male chicken, while the fat pad was reduced by 8.5% in female chicken (table 3, page 2380 and table 4 on page 2381). Rehfeldt et al. (Brit. Poultry Science, 1997, 38:366-373) found that the fat pad was reduced by only 6.0% in male chicken and by 8.5% in female chicken after dosing the animals with racemic clenbuterol (table 1, page 368). Hamano et al. (Brit. Poultry Science 1998, 39: 117-122) also dosed chicken with racemic clenbuterol and could not find any effect of this beta-2 agonist on abdominal fat weight (Table 2, page 119).

New Studies

Two studies have now been performed in broiler chicken. The first study was performed to compare the effects of R-salbutamol and RS-salbutamol with regard to the safety of the drugs in chicken. The second study was performed to investigate the effects of R-salbutamol on body fat. The reason for choosing chicken for the present studies were twofold: Since the safety study had to involve a significant number of animals, there were obvious practical reasons to perform the safety study in broiler chickens rather than mammals. Since the second study involved evaluation of effects on body fat, chicken were chosen because the abdominal fat pad of chicken is easily accessible for measurements.

Studies are also being conducted in obese animals, where the effect of R-salbutamol is compared with the effect of placebo (vehicle) treatment.

1. Safety Study in Chicken.

Three dietary treatments were used in this clinical study. The basal (control) diet was based on maize and soybean meal and was formulated to contain 20% crude protein, 3200 kcal AME/kg, 1.05% lysine and 0.78% methionine+cystine. RS-salbutamol (10 ppm) and R-salbutamol (5 ppm) were incorporated into the control diet to form the two experimental diets. Sixty kilograms of each diet was prepared, and the drugs were mixed into the diets according to the standard operating procedures of the test facility. After mixing, the diets were cold pelleted (65° C.). The diets were analyzed for their concentration of test article immediately after the mixing and at predetermined intervals thereafter. The analytical results confirmed that the sought-after concentrations of the test articles had been obtained and that the “drug-in-feed” was chemically stable.

Day-old male broiler (Cobb) chicken were obtained from a commercial hatchery. The birds were raised in battery brooders housed in an environmentally controlled room and they received a commercial broiler starter diet from day 1 to 12. The birds were transferred to grower cages on day 12. On day 14, birds were weighed individually and birds with relatively high or low body weights were discarded. A total of 72 birds (of uniform body weight) were chosen and distributed into test groups so that average weights per test group were nearly equal. Each of the three dietary treatments (control diet; control diet containing 10 ppm RS-salbutamol; control diet containing 5 ppm R-salbutamol) was then randomly assigned. The diets were fed from day 14 to 28 and the animals were slaughtered on Day 29. Feed and water were available ad libitum. The birds were observed at least three times daily for any unusual behavior or signs. Mortality was recorded daily.

Ethics approval for this study had been obtained from the Animal Ethics Committee of the testing facility.

TABLE 1
Influence of RS- and R-salbutamol on acute toxicity of Cobb broilers,
treated with RS- or R-salbutamol from Day 14 to Day 28
(14 days of treatment).

| | Mortality |
|---|---|
| Treatment | (out of 24 birds) |
| Control | 0/24 |
| 10 ppm RS-salbutamol*) | 2/24 |
| 5 ppm R-salbutamol | 0/24 |

*)10 ppm RS-salbutamol contains 5 ppm R-salbutamol + 5 ppm S-salbutamol

TABLE 2
Tissue concentrations of R- and S-salbutamol in livers from
Cobb broilers, treated with RS- or R-salbutamol from
Day 14 to Day 28 (14 days of treatment).

| | Concentration (ng of drug per g of liver) | | |
|---|---|---|---|
| Treatment | R-salbutamol | S-salbutamol | Total salbutamol |
| Control | 0.0 | 0.0 | 0.0 |
| 10 ppm RS-salbutamol*) | 18.3 | 43.0 | 61.3 |
| 5 ppm R-salbutamol | 23.4 | 0.0 | 23.4 |

*)10 ppm RS-salbutamol contains 5 ppm R-salbutamol + 5 ppm S-salbutamol

Results and Discussion.

Mortality (see Table 1) during this trial occurred only among birds treated with RS-salbutamol. It is believed that the higher mortality in this group is due to the presence of S-salbutamol in these animals.

Importance of this finding: RS-salbutamol in a dose of 10 ppm in the feed caused fatalities. Increasing the dose of RS-salbutamol further will probably cause even higher mortality. There were no fatalities among animals treated with R-salbutamol or vehicle.

Tissue drug levels (see Table 2) have been determined from livers of animals from this study. The concentrations of R-salbutamol after dosing the animals in the feed with 5 ppm R-salbutamol or 10 ppm RS-salbutamol were 18.3 and 23.4 ng/g, respectively. Surprisingly, the tissue concentration of S-salbutamol was approximately twice as high as the concentration of R-salbutamol in the animals treated with RS-salbutamol. This is probably due to a slower metabolism and/or excretion of S-salbutamol than of R-salbutamol by the animals since chicken do not express metabolizing enzymes for R- or S-salbutamol (Fawcett and Aberg, to be published). A stereo-selective delay of the excretion of S-salbutamol is indeed very surprising and has not previously been described for adrenergic beta-2 agonists.

Importance of these findings: It was surprisingly found that animals that were dosed with R-salbutamol had tissue drug concentrations (23.4 ng/g tissue) that were only one third of the tissue drug concentration (61.3 ng/g) in racemate-treated animals. Therefore, the risk for toxic effect in the animals and in humans eating the animals will be very significantly reduced if the animals are dosed with R-salbutamol.

2. Efficacy Study in Chicken.

Three dietary treatments were used in this clinical study. The basal diet (control) diet was based on maize and soybean meal and was formulated to contain 18.9% crude protein, 3200 kcal AME/kg, 1.01% lysine and 0.76% methionine+cystine. R-salbutamol (5 ppm and 10 ppm) was incorporated into the control diet to form the experimental diets. Sixty kilograms of each diet was prepared, and the drugs were mixed into the diets according to the standard operating procedures of the test facility. After mixing, the diets were cold pelleted (65° C.). The diets were analyzed for their concentration of salbutamol immediately after the mixing and at predetermined intervals thereafter. The analytical results confirmed that the sought-after concentrations had been obtained and that the “drug-in-feed” was chemically stable for at least twice the test period.

Day-old male broiler (Cobb) chicken were obtained from a commercial hatchery. The animals were raised in battery brooders housed in an environmentally controlled room and they received a commercial broiler starter diet from day 1 to 14. The birds were transferred to grower cages on day 15 and a commercial broiler finisher diet (18.9% crude protein) was fed. On day 21, birds were weighed individually and birds with relatively high or low body weights were discarded. Animals (of uniform body weight) were distributed into groups of 24 birds each. (Additional treatment groups than those shown here were used and dosed differently.) Each of the dietary treatments was then randomly assigned to the treatment groups. The diets were fed from day 21 and the animals were slaughtered on Day 42. Feed and water were available ad libitum throughout the study. Body weights were recorded at weekly intervals. The animals were observed at least three times daily for any unusual behavior or signs.

Ethics approval for this study had been obtained from the Animal Ethics Committee of the testing facility.

Results:

TABLE 3
Effects of R-salbutamol on body fat and body weight in an animal study

| Group and | Abdominal | Influence on |
|---|---|---|
| oral dose of | fat pad | body weight |
| R-salbutamol | (% BW) | gain (grams) |
| Male broilers | | |
| Control | 1.98 | 1968 |
| 5 ppm | 1.64 | 1886 |
| 10 ppm | 1.63 | 1887 |
| Female broilers | | |
| Control | 2.22 | 1675 |
| 5 ppm | 1.79 | 1646 |
| 10 ppm | 1.77 | 1614 |

N = 24 birds/treatment group.
Weight of the abdominal fat pad is percent of total body weight (BW).
Body weight gain refers to gain during 21 to 42 days of age.

The effects of R-salbutamol on body fat was surprisingly notable and the fat pads in the treated males were decreased by 17.2% and 17.7%, respectively in the animals treated with R-salbutamol in doses of 5 ppm and 10 ppm, respectively. In females, the fat pads were reduced by 19.4% and 20.3%, respectively. The decrease in body weight achieved by R-salbutamol administration was surprising, since other beta-receptor agonists have been shown to increase body weight. Thus, as an example, Dalrymple et al, 1984 (Poultry Science 1984, 63: 2376-2383) reported that body weights of broiler chicken were increased by racemic clenbuterol, which is another adrenergic beta-agonist.

Importance of these findings: It was found that the fat pads in animals that had been treated with R-salbutamol in doses of 5 ppm or 10 ppm in the feed were surprisingly small. This very significant effect on fat tissue development indicates that R-salbutamol not only stimulated lipolysis but also inhibited the hepatic lipogenesis in the animals. Of particular interest is that the fat content was decreased by R-salbutamol, not only in female animals, but there were also very significantly reduction of body fat in male animals. This is in contrast to the teaching in the prior art. Thus, Dalrymple et al. (Poultry Science, 1984, 63: 2376-2383) Zhou and Hahn (Brit. Poultry Science, 1994, 35:355-361) and Rehfeldt et al. (Brit. Poultry Science, 1997, 38:366-373) reported no or minimal effects of racemic beta-2 agonists in male animals. The importance of this finding is particularly important since there is no major use for the enormous amount of fat that is produced by the livestock industry. The production of this fat is enormously costly for the livestock industry and the disposal of the excess fat is both costly and problematic for the industry.

3. Efficacy Study in Obese Young Mammals.

Using commonly known methodology, R-salbutamol is dosed orally to young obese mammals to investigate the effect of the test article on the development of body fat and body weight. Preliminary results indicate that R-salbutamol significantly decreases body fat in obese young animals, thereby demonstrating the usefulness of R-salbutamol in decreasing body fat deposits and inhibiting the development of obesity in mammals.

4. Efficacy Study in Obese Adult Mammals.

Using commonly known methodology, R-salbutamol is dosed orally to adult obese mammals to investigate the effect of the test article on body fat and body weight. Preliminary results indicate that R-salbutamol significantly decreases body fat in obese adult animals, thereby demonstrating the usefulness of R-salbutamol in decreasing deposits of body fat in obese mammals.

### EQUIVALENTS

Those skilled in the art will recognize, or be able to ascertain, using no more than routine experimentation, many equivalents to the specific embodiments of the invention described herein. Such equivalents, include the active isomers also of all other beta-adrenergic agonists, including, but not limited to clenbuterol, salmeterol, terbutaline, fenoterol, formoterol, hexoprenaline, isoprenaline, riniterol, isoetharine, metaproterenol, reproterenol, cimaterol, procaterol, carbuterol, tulobuterol, pibuterol, mabuterol, bitolterol, ractopamine, and bambuterol. All are intended to be encompassed in the scope of the present invention and the following claims. Also included are the eutomers of beta-2 agonists under development, e.g. broxaterol, etanterol, imoxiterol, namiterol, picumeterol, RP 58802, RU 42173, and ZK 90055.

Those skilled in the art will realize that there are many pharmaceutically acceptable salt forms of the drugs of the inventions, such as for example, sulfate, fumarate, hydrobromide, dihydrochloride, methanesulphonate, hydroxynaphthoate, hydrochloride or where appropriate, one or other of the hydrate forms thereof. All such forms are intended to be encompassed in the scope of present invention and the following claims.

1. A method of reducing body fat and/or body weight in obese mammals in need thereof, comprising administering to said obese mammal, a therapeutically effective amount of the eutomeric R-isomer of the adrenergic beta-2 receptor agonist salbutamol or a pharmaceutically acceptable salt thereof, said R-isomer of salbutamol or salt thereof being substantially free of the corresponding distomeric S-isomer.

2. A method of reducing body fat and/or body weight in obese mammals in need thereof, comprising administering to said obese mammal, a therapeutically effective amount of the eutomer of the adrenergic beta-2 receptor agonist salbutamol or a pharmaceutically acceptable salt thereof, while minimizing or eliminating the side effects residing in the corresponding distomeric S-isomer, said R-isomer of salbutamol or salt thereof being substantially free of the corresponding distomeric S-isomer.

3. A method of claim 1 or 2, wherein the optical purity of the eutomer of salbutamol is greater than approximately 85% by weight.

4. A method of claim 1 or 2, wherein the optical purity of the eutomer of salbutamol is greater than approximately 98% by weight.

5. The methods of claim 1 or 2, wherein said mammal is selected from the group consisting of cattle, swine, horses, sheep, deer, dogs, cats and humans.

6. The methods of claim 1 or 2, wherein said eutomer is administered chronically.

7. The method of claim 1 or 2, wherein said mammal is a human.

8. The method of claim 1 or 2, wherein said mammal is a dog.

9. The method of claim 1 or 2, wherein said mammal is a cat.

10. A method of claim 1 or 2, wherein said effective amount of the eutomeric R-isomer of the adrenergic beta-2 receptor agonist salbutamol or a pharmaceutically acceptable salt thereof is mixed with protein-containing food materials.

11. The method of claim 10, wherein the concentration of said R-isomer of salbutamol in said food materials is from approximately 1 ppm to approximately 20 ppm by weight.

12. The method of claim 10, wherein said mammal is administered the optically pure R-isomer of salbutamol orally in the form of an oral administration formulation, containing from approximately 0.5 mg to approximately 5 mg of the optically active R-isomer of salbutamol being substantially free of the corresponding distomeric S-isomer.

13. The method of claim 10, wherein said mammal is administered the optically pure R-isomer of salbutamol parenterally in the form of an administration formulation, containing a daily dose of approximately from 0.5 mg to 5 mg of the optically active R-isomer of salbutamol, substantially free of the corresponding distomeric S-isomer.

14. (canceled)

15. A food composition comprising an admixture of protein-containing food materials with an effective amount of the optically pure R-isomer of the adrenergic beta-2 receptor agonist salbutamol or a pharmaceutically acceptable salt thereof for reducing body fat and/or body weight in a mammal, said food composition being substantially free of the corresponding distomeric S-isomer.
